SEQUENCE LISTING (6 sequences)

SEQ ID NO: 1 - DNA, 24 bp, synthetic construct
  source 1..24: mol_type = other DNA
aagctgaccctgaagttcatctgc

SEQ ID NO: 2 - DNA, 23 bp, synthetic construct
  source 1..23: mol_type = other DNA
cttgtagttgccgtcgtccttga

SEQ ID NO: 3 - DNA, 24 bp, synthetic construct
  source 1..24: mol_type = other DNA
ccggctacaacttgagtcaagtcc

SEQ ID NO: 4 - DNA, 28 bp, synthetic construct
  source 1..28: mol_type = other DNA
cttcatacgggatgatgacatggatgtc

SEQ ID NO: 5 - DNA, 5757 bp, synthetic construct
  source 1..5757: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctagatctaacttgtttattgcagcttataatggttacaaataaggcaatagcatcacaaatttcacaaataaggcatttttttcactgcattctagttttggtttgtccaaactcatcaatgtatcttatcatgtctggatcttacccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacatggtaattcgatgatcccccttaattaatgggccgccgccggccccggagccttttatcgaggcgggcgggagcaccgcccggcccccaggaatgcggccccggccgagggcggacgggacacccaaatatggcgacggcaccattcctcacccgtcgccatatttcggtgtccaccattcctcaccgctctaaaaataactccccgctctaaaaataactcccccaacacctgctgcctgcccaccattcctcaccgctctaaaaataactccccaccattcctcacccgtcgccatatttcggtgtcgtgaggaatggtgccgagggcggacggccgcatgcatttaaataccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtatttaaataccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtggttaattaaaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaagaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcacgcgtggatccaccggtgatccagacatgataagatacattgatgagtttggacaaaccaaaactagaatgcagtgaaaaaaatgccttatttgtgaaatttgtgatgctattgccttatttgtaaccattataagctgcaataaacaagttgatatcgataggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgccagctggcgaaagggggatgtgctgcaaggcgattaagttgggtaacgccagggttttcccagtcacgacgttgtaaaacgacggccagtgccaagctg

SEQ ID NO: 6 - DNA, 6134 bp, synthetic construct
  source 1..6134: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctagatctaacttgtttattgcagcttataatggttacaaataaggcaatagcatcacaaatttcacaaataaggcatttttttcactgcattctagttttggtttgtccaaactcatcaatgtatcttatcatgtctggatcttacccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacatggtaattcgatgatcccccttaattaatgggccgccgccggccccggagccttttatcgaggcgggcgggagcaccgcccggcccccaggaatgcggccccggccgagggcggacgggacacccaaatatggcgacggcaccattcctcacccgtcgccatatttcggtgtccaccattcctcaccgctctaaaaataactccccgctctaaaaataactcccccaacacctgctgcctgcccaccattcctcaccgctctaaaaataactccccaccattcctcacccgtcgccatatttcggtgtcgtgaggaatggtgccgagggcggacggccgcatgcatttaaataccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtatttaaataccagggacagggttatttttagagcgagcttctcctccatggtgtacagagcctaagacccaggcaccggggtggggtaaccgctcaggctcaggcagcaggtgttgggggggggggggcagcaggtgttggggttaattataaccaggcatctcgggtgtccccaggccttgcctccttacatgggcagcctagacccgtagtggttaattaaaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaagaattcgcggccgcacgcgtggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatggccacaaccatggtcttcacactcgaagatttcgttggggactggcgacagacagccggctacaacttgagtcaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggataatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcgtctgcatgaacgcattctggcgggctccggcgaacaaaaactcatctcagaagaggatctgtaaaccggtgatccagacatgataagatacattgatgagtttggacaaaccaaaactagaatgcagtgaaaaaaatgccttatttgtgaaatttgtgatgctattgccttatttgtaaccattataagctgcaataaacaagttgatatcgataggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgccagctggcgaaagggggatgtgctgcaaggcgattaagttgggtaacgccagggttttcccagtcacgacgttgtaaaacgacggccagtgccaagctg

(19) United States
(12) Patent Application Publication
(10) Pub. No.: US 2025/0243510 A1
(43) Pub. Date: Jul. 31, 2025

(54) CONTROLLED MUSCLE-SPECIFIC GENE DELIVERY

(71) Applicant: Rutgers, The State University of New Jersey, New Brunswick, NJ (US)

(72) Inventor: Renping Zhou, Piscataway, NJ (US)

(73) Assignee: Rutgers, The State University of New Jersey, New Brunswick, NJ (US)

(21) Appl. No.: 18/847,288

(22) PCT Filed: Mar. 14, 2023
(86) PCT No.: PCT/US2023/064284

§ 371 Date: Sep. 16, 2024

Related U.S. Application Data
(60) Provisional application No. 63/320,443, filed on Mar. 16, 2022.

(51) Int. Cl. C12N 15/864; A61K 31/65; A61K 48/00; C12N 5/071; C12N 5/073; C12N 5/09; C12N 15/11

(52) U.S. Cl. CPC C12N 15/8645; A61K 31/65; A61K 48/0058; C12N 5/0603; C12N 5/0686; C12N 5/0693; C12N 15/11; C12N 2510/04; C12N 2750/14143; C12N 2750/14145

(57) ABSTRACT

The present invention includes recombinant AAV vectors comprising an inducible transcriptional activator which is operably linked to a muscle-specific promoter, wherein the AAV vector further comprises a capsid gene specific for muscle tissue. The invention further includes methods of producing and using said recombinant AAV vectors to introduce transgenes into target cells and treat diseases in subjects in need thereof.

### CROSS-REFERENCE TO RELATED APPLICATION

The present application claims priority under 35 U.S.C. § 119 (e) to U.S. Provisional Patent Application No. 63/320,443 filed Mar. 16, 2022, which is hereby incorporated by reference in its entirety herein.

### SEQUENCE LISTING

This disclosure contains one or more sequences in a computer readable format in an accompanying.xml file titled “370602-7061WO1 (00167) Sequence Listing ST_26”, which is 17.8 KB in size and was created Mar. 14, 2023, the contents of which are incorporated herein by reference in their entirety.

### BACKGROUND

Adeno associated viral (AAV) vectors have been widely used for gene therapy to transfer therapeutic genes to the target site, in part due to their broad host range, low immunogenicity and cytotoxicity, and long-term expression. In fact, recent studies have demonstrated the efficacy of AAV vectors for correcting single-gene disorders and diseases.

A remaining issue with clinical AAV vectors is off-target transgene expression due to non-specificity in capsid tropism and promoter activity. As such, there is a need for recombinant AAV vectors which offer both higher cell-type specificity and controllable expression for more precise targeted gene therapies. The current invention addresses these needs.

### BRIEF SUMMARY

As described herein, the invention of the present disclosure relates to recombinant AAV vectors comprising an inducible transcriptional activator which is operably linked to a muscle-specific promoter. In certain embodiments, the AAV vector further comprises a capsid gene specific for muscle tissue. In certain aspects, the invention of the present disclosure also includes methods of producing and using said recombinant AAV vectors to introduce transgenes into target cells and treat diseases in subjects in need thereof.

In one aspect, the invention provides a recombinant AAV vector comprising a payload transgene. In certain embodiments, the payload transgene is operably linked to an inducible transcriptional activator. In certain embodiments, the inducible transcriptional activator is operably linked to a muscle-specific promoter. In certain embodiments, the recombinant AAV vector further comprises a capsid gene specific for muscle tissue.

In certain embodiments, the muscle-specific promoter is a hybrid muscle-specific promoter.

In certain embodiments, the hybrid muscle-specific promoter is a modified syn promoter.

In certain embodiments, the modified syn promoter comprises a Mck enhancer.

In certain embodiments, the inducible transcriptional activator is a tetracycline agent-inducible promoter.

In certain embodiments, the tetracycline agent is tetracycline. In certain embodiments, the tetracycline agent is doxycycline. In certain embodiments, the tetracycline agent is anhydrotetracycline.

In certain embodiments, the capsid gene specific for muscle tissue is based on an AAV9 capsid gene.

In another aspect, the invention provides an isolated polynucleotide comprising an AAV vector. In certain embodiments, the vector comprising a payload transgene. In certain embodiments, the payload transgene is operably linked to an inducible transcriptional activator. In certain embodiments, the inducible transcriptional activator is operably linked to a muscle-specific promoter. In certain embodiments, the AAV vector further comprises a capsid gene specific for muscle tissue.

In certain embodiments, the muscle-specific promoter is a hybrid muscle-specific promoter.

In certain embodiments, the hybrid muscle-specific promoter is a modified syn promoter.

In certain embodiments, the modified syn promoter comprises a Mck enhancer.

In certain embodiments, the inducible transcriptional activator is a tetracycline agent-inducible promoter.

In certain embodiments, the tetracycline agent is tetracycline. In certain embodiments, the tetracycline agent is doxycycline. In certain embodiments, the tetracycline agent is anhydrotetracycline.

In certain embodiments, the capsid gene specific for muscle tissue is based on an AAV9 capsid gene.

In another aspect, the invention provides a composition comprising the recombinant AAV vector of the disclosure, and any aspect or embodiment disclosed herein.

In another aspect, the invention provides a method of introducing a transgene into a target cell. In certain embodiments, the method comprises contacting an immortalized cell with the recombinant AAV vector of the disclosure and one or more helper plasmids, thereby producing a packaging cell. In certain embodiments, the method comprises culturing the packaging cell to produce AAV vector particles. In certain embodiments, the method comprises isolating and purifying at least a fraction of the AAV vector particles. In certain embodiments, the method comprises contacting the target cell with an effective amount of the AAV vector particles, thereby introducing the transgene into the target cell. In certain embodiments, the method comprises treating the target cell with an effective amount of a tetracycline agent thereof.

In certain embodiments, the target cell is a muscle cell.

In certain embodiments, the tetracycline agent is tetracycline. In certain embodiments, the tetracycline agent is doxycycline. In certain embodiments, the tetracycline agent is anhydrotetracycline.

In certain embodiments, the immortalized cell is a HEK293 cell. In certain embodiments, the immortalized cell is a HEK293 cell lacking a large T element. In certain embodiments, the immortalized cell is a HeLa cell. In certain embodiments, the immortalized cell is a CHO cell. In certain embodiments, the immortalized cell is a hTERT-immortalized cell.

In another aspect, the invention provides a method of treating, ameliorating, and/or preventing a disease in a subject in need thereof. In certain embodiments, the method comprises administering to the subject an effective amount of the composition of any of the above aspects and embodiments or any aspect or embodiment disclosed herein and further administering to the subject an effective amount of a tetracycline agent.

In certain embodiments, expression of the transgene corrects and/or ameliorates dysfunction of an endogenous gene.

In certain embodiments, the disease is related to dysfunction of an endogenous gene.

In certain embodiments, the tetracycline agent is selected from the group consisting of tetracycline, doxycycline, and anhydrotetracycline.

In certain embodiments, the subject is a mammal.

In certain embodiments, the subject is a human.

### BRIEF DESCRIPTION OF THE DRAWINGS

The following detailed description of specific embodiments of the invention will be better understood when read in conjunction with the appended drawings. For the purpose of illustrating the invention, exemplary embodiments are shown in the drawings. It should be understood, however, that the invention is not limited to the precise arrangements and instrumentalities of the embodiments shown in the drawings.

FIGS. 1A-1B illustrate muscle cell-oriented AAV serotype selection. (FIG. 1A) AAV2-CMV-eGFP-MTP, AAV6-CMV-eG/FP, AAV9-CMV-eGFP infected 293T and differentiated C2C12 cells. Cells were cultured in 6-well plates and infected by 2×10⁷ vg each well. Images of 293T and C2C12 cells were taken 3 and 5 days after viral infection respectively. All images were taken by same magnification and exposure time. Scale bars are 200 μm. (FIG. 1B) Integrated density analysis of GFP signal. Integrated density was measured by software Image J. All data were averaged from three separate fields.

FIGS. 2A-2C illustrate muscle specific vector design and in vitro testing. (FIG. 2A) Structure diagram of the muscle specific AAV9 vector. ITR, inverted terminal repeat; SV40, simian virus 40 promotor; rtTA, reverse tetracycline-controlled trans-activator; MCK, muscle creatine kinase enhancer; Dox, doxycycline; TRE3GS, third-generation tet-responsive promoter; teLuc, a modified nanoluciferase. (FIG. 2B) GFP expression of the muscle specific vector and CMV control vector. Scale bars are 200 μm. (FIG. 2C) teLuc activity assay. AU/μg, arbitrary unit per microgame of protein. Each data represents the mean±SD value of triplicate experiments. The p-value was determined using Two-tailed t-Test. *** P<0.001 versus CMV group.

FIG. 3 illustrates bioluminescence imaging of hairless mice after IV viral injection. Adult mice were injected with 1×10¹⁴ vg/kg viruses via retro-orbital administration. Images were taken 1 month after viral injection via in vivo imaging system (IVIS). Diphenylterazine (DTZ) was injected 5 minutes before imaging. Viruses injected were (FIG. 3A) AAV-CMV-Teluc, (FIG. 3B) AAV-MCK-TetOn-teLuc with Dox induction, (FIG. 3C) AAV-MCK-TetOn-teLuc without Dox induction. The luminescence value was 7264, 5923, and 243 respectively.

FIGS. 4A-4C illustrate the luciferase activity of virus injected animal tissue samples. Heart, lung, diaphragm, liver, kidney, biceps femoris, biceps brachii muscles, and brain tissue were collected one month after viral injection and induction. Luciferase activities were assayed with substrate DTZ. Data are mean+SD, n=3 each group (FIG. 4A) Luciferase activity of animal tissues after AAV-CMV-teLuc injection, (FIG. 4B) luciferase activity of tissue sample of AAV-MCK-Tet-teLuc injection with and without Dox induction. (FIG. 4C) virus gene copy number analyzed by qPCR. Data are mean+SD.

FIGS. 5A-5D illustrate the relative expression of viral genes. Animal tissue luciferase activity were normalized by virus gene copy number extracted from the same pieces of tissue. (FIG. 5A) Virus gene relative activity of animal tissue after AAV-CMV-teLuc injection, Data are mean+SD (FIG. 5B) Virus gene relative activity of animal tissue after AAV-MCK-Tet-teLuc injection with and without Dox induction, Data are mean+SD (FIG. 5C) calculation of virus gene relative expression level. Bioluminescence were normalized by total protein concentration and viral gene copy number were normalized by total genomic DNA amount. (FIG. 5D) CMV and MCK groups viral gene expression level are presented as normalization of the value from heart tissue. Data are mean+SD, n=3. p-value was determined by two-tailed t-test. * p<0.01 versus CMV group.

FIGS. 6A-6D illustrate GFP expression in muscle tissue following viral injection. AAV9-MCK-Tet-GFP and AAV9-CMV-GFP were injected 1×10¹⁴ vg/kg intramuscularly. Muscle tissue were collected after 1 month of viral injection and induction, frozen sections were stained with DAPI (300 nM) and florescent image (GFP) were taken via Nikon NIS-Elements platform. (FIG. 6A) muscle tissue without viral injection (100 ms), (FIG. 6B) AAV-MCK-Tet-GFP with dox induction (100 ms), (FIG. 6C) muscle tissue without viral injection (5 ms), (FIG. 6D) AAV-CMV-GFP injection (5 ms).

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a”, “an”, and “the” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “AAV vector” as used herein refers to a polynucleotide vector comprising one or more genes of interest (or transgenes) and associated promoters and enhancers that are flanked by AAV terminal repeat sequences (ITRs). AAV vectors can be produced and packaged into infectious viral particles when present in a host cell that has been transfected with one or more helper plasmids encoding and expressing rep and cap proteins and one or more proteins from adenovirus open reading frame E4orf6. The AAV vectors may be operably linked to promoter and enhancer sequences that can regulate the expression of the protein encoded by the AAV vector.

The terms “AAV virion” or “AAV viral particle” or “AAV vector particle” as used herein refers to a viral particle composed of capsid proteins from at least one AAV serotype surrounding a polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

The term “packaging” as used herein refers to intracellular process by which viral virions or particles (e.g. AAV virions or particles), especially viral vector particles or virions are assembled in a host cell. “Packaging” cells comprise the cells that contain the polynucleotide (e.g. helper plasmids) and protein components necessary to assemble functional viral virions.

A “biomarker” or “marker” as used herein generally refers to a nucleic acid molecule, clinical indicator, protein, or other analyte that is associated with a disease. In certain embodiments, a nucleic acid biomarker is indicative of the presence in a sample of a pathogenic organism, including but not limited to, viruses, viroids, bacteria, fungi, helminths, and protozoa. In various embodiments, a marker is differentially present in a biological sample obtained from a subject having or at risk of developing a disease (e.g., an infectious disease) relative to a reference. A marker is differentially present if the mean or median level of the biomarker present in the sample is statistically different from the level present in a reference. A reference level may be, for example, the level present in an environmental sample obtained from a clean or uncontaminated source. A reference level may be, for example, the level present in a sample obtained from a healthy control subject or the level obtained from the subject at an earlier timepoint, i.e., prior to treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. The differential presence of a marker of the invention in a subject sample can be useful in characterizing the subject as having or at risk of developing a disease (e.g., an infectious disease), for determining the prognosis of the subject, for evaluating therapeutic efficacy, or for selecting a treatment regimen.

By “agent” is meant any nucleic acid molecule, small molecule chemical compound, antibody, or polypeptide, or fragments thereof.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

By “fragment” is meant a portion of a nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 10, 15, 20, 30, 40, 50, 60, 70, 80, 90, or 100 nucleotides.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Hybridization” means hydrogen bonding, which may be Watson-Crick, Hoogsteen or reversed Hoogsteen hydrogen bonding, between complementary nucleobases. For example, adenine and thymine are complementary nucleotides that pair through the formation of hydrogen bonds.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “marker profile” is meant a characterization of the signal, level, expression or expression level of two or more markers (e.g., polynucleotides).

By the term “microbe” is meant any and all organisms classed within the commonly used term “microbiology,” including but not limited to, bacteria, viruses, fungi and parasites.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

As used herein, the term “operably linked” refers to a promoter and/or other genetic regulatory sequence(s) which is positioned relative to a nucleic acid sequence encoding a gene in such a way as to direct, influence, or regulate expression of the gene. A regulatory sequence can be “operably linked” with a gene sequence in the same vector or in a different vector. One or more regulatory sequences operably linked to a gene sequence can be contiguous and/or can act in trans or at a distance to direct, influence or regulate expression of the gene. Among the regulatory sequences, a promoter is essential, while other optional regulatory sequences such enhancers, introns, and terminators can improve expression or control of expression.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

“Parenteral” administration of an immunogenic composition includes, e.g., subcutaneous (s.c.), intravenous (i.v.), intramuscular (i.m.), or intrasternal injection, or infusion techniques.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By “reference” is meant a standard of comparison. As is apparent to one skilled in the art, an appropriate reference is where an element is changed in order to determine the effect of the element. In one embodiment, the level of a target nucleic acid molecule present in a sample may be compared to the level of the target nucleic acid molecule present in a clean or uncontaminated sample. For example, the level of a target nucleic acid molecule present in a sample may be compared to the level of the target nucleic acid molecule present in a corresponding healthy cell or tissue or in a diseased cell or tissue (e.g., a cell or tissue derived from a subject having a disease, disorder, or condition).

As used herein, the term “sample” includes a biologic sample such as any tissue, cell, fluid, or other material derived from an organism.

By “specifically binds” is meant a compound (e.g., nucleic acid probe or primer) that recognizes and binds a molecule (e.g., a nucleic acid biomarker), but which does not substantially recognize and bind other molecules in a sample, for example, a biological sample.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95%, 96%, 97%, 98%, or even 99% or more identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “subject” is meant a mammal, including, but not limited to, a human or non-human mammal, such as a bovine, equine, canine, ovine, feline, mouse, or monkey. The term “subject” may refer to an animal, which is the object of treatment, observation, or experiment (e.g., a patient).

By “target nucleic acid molecule” is meant a polynucleotide to be analyzed. Such polynucleotide may be a sense or antisense strand of the target sequence. The term “target nucleic acid molecule” also refers to amplicons of the original target sequence. In various embodiments, the target nucleic acid molecule is one or more nucleic acid biomarkers.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “tetracycline agent” refers to a tetracycline, doxycycline, anhydrotetracycline, derivative thereof, and/or analogue thereof.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based in certain aspects on the unexpected observation that certain regulatory elements can be combined into AAV vectors which allow for both temporal and tissue-specific control of their payload transgenes. These regulatory elements include a combination of particular capsid proteins with general tissue specifies combined with a tissue-specific promoter and enhancer driving the expression of an inducible transcription activator. As such, the combination of these three control aspects allow for the precise timing and tissue-specific control of transgene expression. This process enables more efficacious gene therapy by reducing off-target and inappropriate transgene expression. Also included are isolated nucleic acids comprising the AAV vectors and method of introducing transgenes into target cells using said AAV vectors.

### AAV Vectors

AAV are relatively small, non-enveloped viruses with a ˜4 kb genome that is flanked by inverted terminal repeats (ITRs). The genome contains two open reading frames, one of which provides proteins necessary for replication and the other provides components required for construction of the viral capsid. Wild-type AAV is typically found in the presence of adenovirus as the adenoviruses provide helper proteins that are essential for packaging of the AAV genome into virions. Therefore, AAV production piggy-backs on co-infection with adenovirus and relies on three key elements: the ITR-flanked genome, the open-reading frames, and adeno-helper genes. Due to their ability to readily infect human cells without causing apparent disease, AAV is well-studied as a vector for gene delivery. AAV may be readily obtained and their use as vectors for gene delivery has been described in, for example, Muzyczka, 1992; U.S. Pat. No. 4,797,368, and PCT Publication WO 91/18088. Construction of AAV vectors is described in a number of publications, including Lebkowski et al., 1988; Tratschin et al., 1985; Hermonat and Muzyczka, 1984; vectors is described in a number of publications, including Lebkowski et al., 1988; Tratschin et al., 1985; Hermonat and Muzyczka, 1984.

AAV-based vector systems typically separate the viral AAV genes, Adenovirus-derived helper genes, and the transgene payload onto two or three separate plasmids. Three plasmid systems consist of an AAV helper plasmid comprising the rep (replication) and cap (capsid) genes, an adenoviral helper plasmid comprising at least the E2a gene, E4 gene, and VA (viral associated) RNA, and a payload plasmid comprising the transgene and associated promoters and enhancers flanked by ITR sequences. The helper plasmid or plasmids do not comprise ITRs in order to prevent packaging of a functional, infectious viral genome. Two plasmid systems combine the AAV rep and cap genes and adenoviral helper genes onto a single plasmid and simplify viral vector production by reducing the number of transfected plasmids. Often, a dedicated packaging cell line is used which is engineered to express AAV/helper genes prior to introduction of the payload plasmid.

In certain embodiments, the plasmid comprising the payload transgene also comprises enhancers and transcriptional activators which precisely control the tissue location and timing of expression of the transgene. In certain embodiments, the enhancer is a hybrid promoter/enhancer derived from the muscle creatine kinase (Mck) gene which contains muscle cell-specific transcription binding sites. In certain embodiments, the hybrid Mck enhancer is operably linked to an inducible transcriptional activator which is active in the presence of a small-molecule ligand. In certain embodiments, the transcriptional activator is activated by a tetracycline, doxycycline, anhydrotetracycline, derivative thereof, and/or analogue thereof (generally referred to as “tetracycline agent” herein) and is a so-called “TetON” activator. In this way, expression of the payload transgene only occurs in the presence of both Mck-binding transcription factors (i.e. in a muscle cell) and the presence of a tetracycline agent.

### Tissue Targeting of AAVs

Successful gene therapies require efficient infection of target tissues and establishment of long term gene expression, and previous studies have demonstrated that AAV vectors can successfully infect and transduce a broad variety of cell and tissue types, such as brain, liver, muscle, among others, and has the ability to infect both dividing and quiescent cells. Additionally, AAV-mediated transduction of tissues has been demonstrated to result in long term gene expression greater than 1.5 years in animal models including canine, murine and hamster.

The tissue tropism of AAV vector particles is influenced by the serotype of the capsid protein, though the receptors and co-receptors that the capsid proteins bind to are often poorly understood and can be expressed by multiple tissue types. For example, AAV2, one of the most well-studied serotypes, has a binding affinity largely for heparan sulfate proteoglycan (HSPG) and as such has a tropism in humans for eye, brain, lung, liver, muscle, and joint tissues. Likewise, AAVs 1, 4, 5, and 6 have a binding affinity largely for sialic acid and a tropism for neuronal tissues and AAVs 5,8, and 9 which share a tropism for skeletal muscle cell among other tissues. In this way, the serotype of the AAV capsid protein can be selected to target the payload nucleic acid of the AAV vector to a specific tissue or cell type. Alteration or modification of capsid protein structure can also alter the tissue or cellular tropism and affinity of the resulting AAV vector particles.

In certain embodiments, the current invention comprises helper plasmids comprising a capsid protein derived from AAV9 and variants thereof. AAV9 and capsid proteins based on AAV9 have a tropism for multiple tissue types including heart and skeletal muscle, CNS, lung, and liver. In the AAV vectors of the current invention, the combination of AAV9 capsid muscle cell tropism with a skeletal muscle-specific Mck hybrid enhancer/promoter restricts expression of the payload transgene to skeletal muscle cells. It is also contemplated that the invention could be used with any capsid protein capable of selectively binding to skeletal muscle cells, including but not limited to engineered, or otherwise modified, hybrid, or synthetic capsid proteins. In certain embodiments, these engineered capsid proteins can be based on or derived from AAV9 or any other capsid protein with a binding affinity for muscle cells. In certain embodiments, the AAV vectors of the invention can be used with any naturally occurring, modified, hybrid, or engineered AAV capsid protein including, but not limited to AAV1, AAV2, AAV3, AAV3B, AAV4, AAV5, AAV6, AAV7, AAV8, AAV9, AAV12, AAV-B1, AAV-DJ, AAV-Retro, AAVrh8, AAVrh10, AAVrh25, Anc80L65, LK03, AAVrh18, AAVrh74, AAVrh32.33, AAVrh39, AAVrh43, Oligo001, PHP-B, and Spark100 among others. The skilled artisan would be able to select an appropriate capsid protein for use with the invention based on the desired target tissue or cell type.

### Transcriptional Control of Payload Genes

In certain embodiments, the current invention includes recombinant AAV vectors which incorporate temporal and tissue-specific control features which restrict expression of the transgene payload. In certain embodiments, the recombinant AAV vectors comprise muscle-specific hybrid enhancer/promoters which are derived from the genomic elements that control expression of the muscle creatine kinase (Mck) gene. Also called an “E-Syn” hybrid promoter, this synthetic element is a combination of Mck enhancer and promoter sequences, which can efficiently drive gene expression in both skeletal and heart tissue, while its 520 bp size is compact enough for use in recombinant AAV vectors, which are typically size-limited (Wang, et al. (2008) Gene Therapy, 15, 1498-1499).

In another aspect, the current invention includes recombinant AAV vectors which comprise an inducible transcriptional activator which is operably linked to the payload transgene. In certain embodiments, the inducible transcriptional activator is activated by the presence of doxycycline, tetracycline, anhydrotetracycline, or a similar molecule (“tetracycline agent”). So-called “Tet-On” gene expression systems allow for the precise control of expression of genes of interest. Tet technology comprises two complementary control circuits, now commonly referred to as the Tet-Off system (tTA dependent) and the Tet-On system (rtTA dependent). In each system, a recombinant tetracycline agent-controlled transcription factor (tTA or rtTA) interacts with a tTA/rtTA responsive promoter, Ptet, to drive expression of the gene of interest. Expression is regulated by presence of tetracycline, doxycycline, anhydrotetracycline, or one of its derivatives.

Tetracycline agents act at the level of DNA binding of tetracycline agent-controlled transactivator (tTA) and reverse tetracycline agent-controlled transactivator (rtTA) transcription factors. rtTA requires a tetracycline agent ligand for DNA binding and transcription. By contrast, the interaction between tTA and DNA is prevented by tetracycline agents. Thus, the two versions of the Tet system respond to tetracycline agents differently and may be used in a complementary manner. In certain embodiments of the current invention, the muscle-specific hybrid promoter drives expression of the rtTA, which activates transcription of payload transgenes when in the presence of a tetracycline agent. In this way, a recombinant AAV vector of the current invention can be administered to a subject followed by administration of tetracycline, doxycycline, anhydrotetracycline, or a derivative there which expresses the payload transgene only in muscle tissue and only for the duration of a tetracyclic agent. In certain embodiments, the recombinant AAV vectors of the invention comprise so-called third generation or 3G inducible tetracycline activators which comprise enhanced promoters with significantly reduced background expression and enhanced transactivator proteins with increased sensitivity for a tetracycline agent as compared to previous versions of these systems.

### Methods of Use

In another aspect, the invention provides a method of introducing a transgene into a target cell, comprising contacting an immortalized cell with the recombinant AAV vector of any one of the invention and one or more helper plasmids, thereby producing a packaging cell, culturing the packaging cell to produce AAV vector particles, isolating and purifying the AAV vector particles, contacting the target cell with an effective amount of the AAV vector particles, thereby introducing the transgene into the target cell, and treating the target cell with an effective amount of a tetracycline agent.

In certain embodiments, the immortalized cell already comprises the helper and adenoviral genes necessary for the generation of AAV vector particles. A number of cell lines capable of being used as packaging cells for AAV vectors are known in the art and are contemplated for use with the recombinant AAV vectors of the invention to produce AAV particles, including but not limited to HEK293 cells, HEK293 cells lacking a large T element, HeLa cells, CHO cells, and hTERT-immortalized cells, among others. In certain embodiments, the target cell is a muscle cell. In certain embodiments, the tetracycline agent is selected from the group consisting of tetracycline, doxycycline, anhydrotetracycline, or any derivative thereof.

In another aspect, the invention includes a method of treating a disease in a subject in need thereof, comprising administering to the subject an effective amount of a composition comprising the recombinant AAV vectors of the invention and a pharmaceutically acceptable diluent or excipient followed by an effective amount of a tetracycline agent. In certain embodiments, the expression of the transgene corrects the dysfunction of an endogenous gene. In certain embodiments, the disease is related to a dysfunction of an endogenous gene. In certain embodiments, the subject is a mammal. In certain embodiments, the subject is a human. In certain embodiments, the tetracycline agent is selected from the group consisting of tetracycline, doxycycline, anhydrotetracycline and a derivative thereof.

The tissue specificity of the recombinant AAV vectors of the invention make them particularly useful for the treatment of neuro-muscle-related diseases or diseases resulting from abnormalities or mutations in muscle cells. As such in certain embodiments, the disease is a muscular dystrophy including ALS, Duchenne, Becker, tibial, congenital, oculopharyngeal, fascioscapulohumeral, myotonic, Emery-Dreifuss, and limb-girdle, Miyoshi myopathy, Welander myopathy, Nonakel myopathy, Laing myopathy, a myofibrillary/desmin-related myopathy, epidermolysis bullosa, Leber congenital amaurosis, spinal muscular atrophy, X-linked myotubular myopathy, and a gene-related diseases which occurs in muscle cells such as Pompe disease.

In certain embodiments, the method of the invention comprises harvesting and purifying the AAV vector particles. A number of techniques are known in the art for the efficient purification of AAV particles including, but not limited to centrifugation over a gradient and chromatography. In certain embodiments, the centrifugation occurs at very high speeds and is also known as ultracentrifugation or ultrahigh speed centrifugation. Purification methods employing centrifugation often include the use of a gradient in which the separation of viral particles is based on the density of the particles in a medium of varying density. A number of different density mediums are known in the art including but not limited to cesium chloride (CsCl), sepharose-based medium, and iodixanol including OptiPrep™ Chromatography-based purification methods include, but are not limited to affinity based, heparin based, and ion exchange techniques. Affinity based techniques make use of antibodies specific for the assembled AAV capsid. Heparin-based techniques take advantage that AAV particles bind to heparin sulfate proteoglycan with high affinity. In certain embodiments, the purification method involves a chemical partitioning technique including an aqueous two-phase system. In certain embodiments, the purification technique involves any combination of these techniques.

### Pharmaceutical Compositions

Certain embodiments of the disclosure are directed to therapeutically treating an individual in need thereof. As used herein, the term “therapeutically” includes, but is not limited to, the administration of a treatment comprising a recombinant AAV vector of the invention particle to a subject who displays symptoms or signs of pathology, disease, or disorder, in which treatment is administered to the subject for the purpose of diminishing or eliminating those signs or symptoms of pathology, disease, or disorder.

As used herein, the term “subject” is intended to include living organisms such as mammals. Examples of subjects include, but are not limited to, horses, cows, sheep, pigs, goats, dogs, cats, rabbits, guinea pigs, rats, mice, gerbils, non-human primates, humans and the like, non-mammals, including, e.g., non-mammalian vertebrates, such as birds (e.g., chickens or ducks) fish or frogs (e.g., Xenopus), and a non-mammalian invertebrates, as well as transgenic species thereof. Preferably, the subject is a human.

Pharmaceutical compositions of the present disclosure may comprise the therapeutic engineered recombinant AAV vector particles as described herein, in combination with one or more pharmaceutically or physiologically acceptable carriers, diluents or excipients. Such compositions may comprise buffers such as neutral buffered saline, phosphate-buffered saline (PBS) and the like; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for a number of administration routes including oral, inhalation, nasal, nebulization, intravenous injection, intramuscular injection, intrathecal injection, intrapleural injection, intracisternal magna injection, subcutaneous injection, and/or transdermal injection. Pharmaceutical compositions of the present disclosure may be administered in a manner appropriate to the disease to be treated (or prevented). The quantity and frequency of administration will be determined by such factors as the condition of the patient, the type and severity of the patient's disease, and the type and functional nature of the patient's immune response to the phage particles, although appropriate dosages may be determined by clinical trials.

The recombinant AAV vector particles of the disclosure can be administered in dosages and routes and at times to be determined in appropriate pre-clinical and clinical experimentation and trials. Administration of the recombinant AAV vector particles of the disclosure may be combined with other methods useful to treat the desired disease or condition as determined by those of skill in the art.

In certain embodiments, the effective dose range is measured in units known to a person of skill in the art to be suitable for the description of recombinant AAV vector particle doses. In some embodiments, the effective dose range for a vaccine or therapeutic compound of the disclosure is measured by transducing units (TU)/kg/dose or genome copies (GC)/kg/dose or particles/kg/dose. In some embodiments, the dosage provided to a patient is between about 10⁶-10¹⁴ TU/kg. In some embodiments, the dosage provided to a patient is between about 10⁶-10¹⁴ GC/kg. In some embodiments, the effective dose range is measured by colony forming units (CFU), 50% tissue culture infectious dose (TCID₅₀), and combinations thereof.

Actual dosage levels of the active ingredients in the pharmaceutical compositions of this disclosure can be varied so as to obtain an amount of the active ingredient that is effective to achieve the desired therapeutic response for a particular patient, composition, and mode of administration, without being toxic to the patient.

The therapeutically effective amount or dose of a compound of the present disclosure depends on the age, sex and weight of the patient, the current medical condition of the patient and the progression of a disease or disorder contemplated in the disclosure.

A medical doctor, e.g., physician or veterinarian, having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Dosage size can be adjusted according to the weight, age, and stage of the disease of the subject being treated. Recombinant AAV vector particles may also be administered multiple times at these dosages. The recombinant AAV vector particles can be administered by using infusion techniques that are commonly known in the art of immunotherapy or vaccinology. The optimal dosage and treatment regime for a particular patient can readily be determined by one skilled in the art of medicine by monitoring the patient for signs of disease and adjusting the treatment accordingly.

The administration of the recombinant AAV vector particle compositions of the disclosure may be carried out in any convenient manner known to those of skill in the art. The recombinant AAV vector particles of the present disclosure may be administered to a subject by aerosol inhalation, injection, ingestion, transfusion, implantation or transplantation. The compositions described herein may be administered to a subject or patient trans-arterially, subcutaneously, intranasally, intradermally, intratumorally, intranodally, intramedullary, intramuscularly, intravenously, or intraperitoneally. In other instances, the recombinant AAV vector particles of the disclosure are injected directly into a site of inflammation in the subject, a local disease site in the subject, a lymph node, an organ, a tumor, and the like. It should be understood that the method and compositions that would be useful in the present disclosure are not limited to the particular formulations set forth in the examples.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier can be a solvent or dispersion medium containing, for example, saline, buffered saline, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it is advisable to include isotonic agents, for example, sugars, sodium chloride, or polyalcohols such as mannitol and sorbitol, in the composition.

Formulations can be employed in admixtures with conventional excipients, i.e., pharmaceutically acceptable organic or inorganic carrier substances suitable for any suitable mode of administration, known to the art. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., analgesic agents.

The practice of the present disclosure employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, fourth edition (Sambrook, 2012); 30 “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and AAV particles of the disclosure, and, as such, may be considered in making and practicing the disclosure. It should be understood that the method and compositions that would be useful in the present disclosure are not limited to the particular formulations set forth in the examples. The following examples are put forth so as to provide those of ordinary skill in the art with a complete disclosure and description of how to make and use the cells, expansion and culture methods, and therapeutic methods of the disclosure, and are not intended to limit the scope of what the inventors regard as their disclosure.

### EXPERIMENTAL EXAMPLES

The disclosure is now described with reference to the following Examples. These Examples are provided for the purpose of illustration only, and the disclosure is not limited to these Examples, but rather encompasses all variations that are evident as a result of the teachings provided herein.

The materials and methods used in the following experimental examples is now provided.

Recombinant AAV vectors. AAV-P_{CMV}-eGFP was obtained from Addgene (Cat. #67634; Xiong et al., 2015). AAV-P_{CMV}-teLuc was generated by replacing the eGFP gene with a teLuc gene PCR fragment using pcDNA3-teLuc c-myc (Addgene Cat. 100026; Yeh et al Nat Methods, 2017) as template, using In-Fusion ligation (Takara Bio, Cat. 638956). To construct AAV-Pmus-TetOn vectors, a DNA fragment containing XhoI-Transcription Blocker sequences-two copies of the Mck enhancer element-C5-12 synthetic promoter (Li et al., 1999)-XbaI, was synthesized (Genscript Biotech Corp, NJ) and used to replace the XhoI-XbaI fragment containing the transcription blocker and hPGK promoter in pAAVpro-Tet-One plasmid (Takara Bio Cat. #634311), generating pAAV-Pmus-TetOn. pAAV-Pmus-TetOn-eGFP was constructed by ligating the EcoRI-NotI eGFP DNA fragment from pEGFP-N1 plasmid (Clontech Cat. #6085-1; Genbank accession #U55762.1) into the EcoRI-NotI sites of the pAAV-Pmus-TetOn plasmid. pAAV-Pmus-TetOn-teLuc was constructed by ligating a PCR teLuc fragment using pcDNA3-teLuc c-myc plasmid as a template into the NheI-BamHI sites of pAAV-Pmus-TetOn downstream of the TRE3GS promoter. In addition, a chimeric intron from pCI-Neo plasmid (Promega, Cat. #E1841; Genbank Accession #U47120) was inserted between the TRE3GS promoter and teLuc gene.

Virus production and purification. Purified AAVs were prepared based on published protocols (Challis et al., 2016). Briefly, 70%-80% confluence LentiX-293T cells (Takara Bio) grown in DMEM supplemented with 10% FBS, NEAA, Sodium Pyruvate, and Sodium Bicarbonate were triple transfected with recombinant AAV, pHelper, and pUCmini-iCap-PHP.eb (or AAV2-MTP and pMDG6 for AAV2 and 6 respectively). At day 1 and day 3 post transfection, medium was refreshed with supplement of 4% FBS, NEAA, Sodium Pyruvate, and Sodium Bicarbonate. At day 5 post transfection, medium (together with day 3 medium) and cells were collected and processed separately. Cells were lysed through multiple freeze and thaw cycles; supernatants after centrifugation were collected for purification. Viral particles in the culture medium were precipitated out by polyethylene glycol (PEG) 8000 and followed by centrifugation. Concentrated AAVs were purified by CsCl gradient ultracentrifugation. For smaller scale AAV production, viruses were collected by the AAVpro Extraction Kit (Takara Bio) for in vitro use. Virus titers were determined by qPCR with the following primers: eGFP+, 5′-AAGCTGACCCTGAAGTTCATCTGC-3′ (SEQ ID NO: 1); eGFP-, 5′-CTTGTAGTTGCCGTCGTCCTTGA A-3′ (SEQ ID NO: 2); teluc+, 5′-CCGGCTAC AACTTGAGTCAAGTCC-3′ (SEQ ID NO: 3); and teluc-, 5′-CTTCATACGGGATGAT GACATGGATGTC-3′ (SEQ ID NO: 4).

Cell culture. HEK 293T cells were cultured in high glucose DMEM with 10% FBS and penicillin/streptomycin. C2C12 cells were kept in DMEM with 10% FBS, the differentiation medium used 2% horse serum in substitution of 10% FBS. C2C12 myotubes were infected at differentiation day 4. For cells infected with AAV-Pmus-TetOn-eGFP/teLuc, the tissue culture medium was supplemented with 1 mg/ml doxycycline.

Luciferase assay. Cell or tissue were lysed in NP-40 buffer with proteinase inhibitor for 30 minutes on ice, supernatants were collected after centrifuge at 1,800×g for 20 minutes. Diphenylterazine (DTZ) were used as luciferase substrate. DTZ were dissolved in in vivo luciferase assay buffer which contains 8% glycerol (v/v), 10% ethanol (v/v), 10% Hydroxypropyl-β-cyclodextrin (m/v), 35% PEG 400 (v/v) and water. For in vitro luciferase assays, DTZ solution were diluted in the in vitro luciferase buffer (1 mM CDTA, 0.5% tergitol NP-40, 0.05% antifoam 204, 150 mM KCl, 100 mM MES (PH 6.0), 35 mM thiourea). 20 ul cell or tissue lysates were mixed with 100 ul DTZ in in vitro buffer for bioluminescence detection in 96-well plates.

Animal studies. C57/b6 and hairless mice were used in this study. 100 ul of AAV solution were injected retro-orbitally following ketamine/xylazine anesthesia. If gene expression induction is needed, 625 mg/kg Dox diet (Envigo) were provided. Mice were anesthetized by isoflurane inhalation. 100 ul DTZ in vivo solution (2.5 mg/ml) were injected subcutaneously 5 minutes before imaging. Images were taken with IVIS Spectrum, PerkinElmer Co., Ltd. The exposure time was 2 minutes.

Animals were sacrificed before tissue dissection. Tissues were lysed in NP-40 buffer for luciferase assay as described above. Tissue genomic and viral DNA were extracted by Monarch Genomic DNA Extraction Kit (New England Biolabs) per manufacture's instruction. DNA concentrations were measured by NanoDrop 2000 (Thermo Fisher). Tissue viral gene copy number were determined by qPCR.

### Example 1: Identifying AAV Serotype Preferentially Targeting Muscle Cells

AAV serotype 2 (AAV2) is the first isolated serotype and most widely used in AAV based gene therapy studies. AAV2 moderately transduce several tissues including the central nerves system (CNS), liver, muscle, and lung. To increase muscle tropism, a 7 amino acid MTP has been inserted in the capsid of AAV2 (AAV2-MTP) as previously described. AAV6 has been reported to have a better transduction rate in skeletal muscles, and AAV9 is reported to target liver, heart, skeletal muscles, and CNS more efficiently. AAV2-MTP, AAV6 and AAV9 were tested in vitro to assess their ability to infect muscle cells.

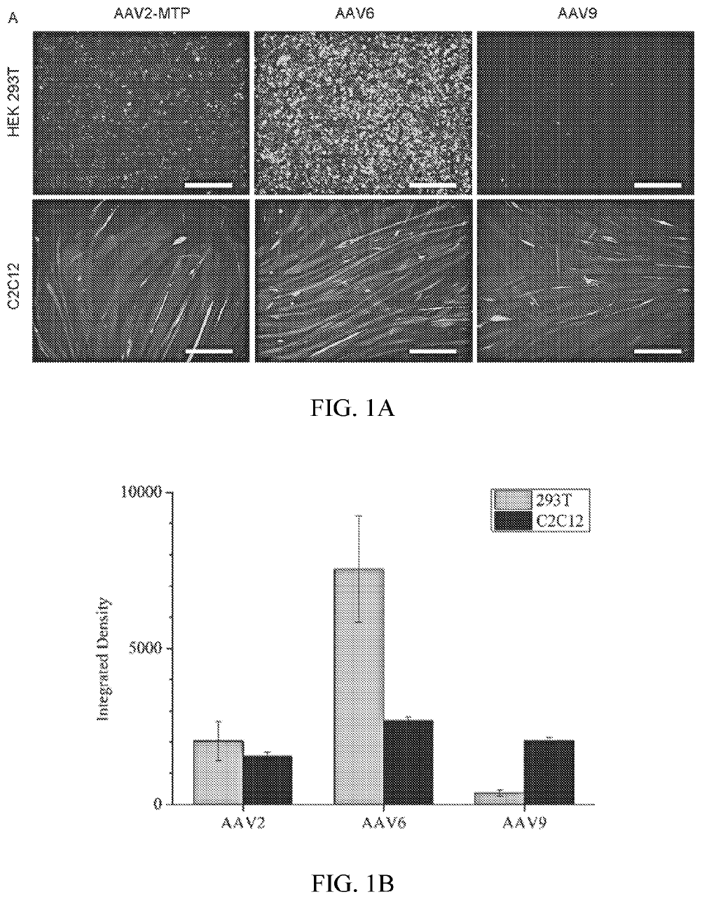

HEK 293T and differentiated C2C12 cells were infected with the same amount of AAV2-MTP-CMV-eGFP, AAV6-CMV-eGFP, and AAV9-CMV-eGFP, expression levels were determined by GFP florescence intensity from the images taken (FIG. 1). All 3 vectors showed comparable GFP expression level in differentiated C2C12 cells, however, AAV6 had at a much higher expression level in HEK 293T than in C2C12 cells, which failed to show muscle tissue-specific expression. AAV2-MTP had lower expression level in HEK 293T cells than AAV6, but there was no significant difference between the two cell lines. AAV9 showed the least GFP expression in HEK 293T cells compared to AAV2-MTP and AAV6, thus was used for further studies.

### Example 2: Designing a Spatially and Temporally Regulatable Vector for Gene Delivery in Muscle Cells

To construct a spatially and temporally regulatable AAV gene delivery vector, we choose to use the hybrid muscle promoter (Pₘᵤₛ) to drive the expression of the tetracycline agent-controlled transcriptional activator TetOn3G, which in turn transcribes gene of interest under the control of a tetracycline agent response element promoter P_{TRE3GS}, which contains 7 tetracycline operator (TetO) sequences and a CMV minimal promoter, in the presence of doxycycline (Dox) (FIG. 1A?). P_{TRE3GS} was a modified version of the original P_{TRE} to eliminate binding sites for endogenous mammalian transcription factors to lower background expression. Here we used eGFP and teLuc (a modified nano-luciferase with red-shifted high bioluminescence; Yeh et al., 2017) as reporter genes. The vectors are named as AAV9-Pₘᵤₛ-TetOn-eGFP/teLuc and the constructs were tested both in vitro and in vivo.

### Example 3: Testing Recombinant AAV Vector In Vitro

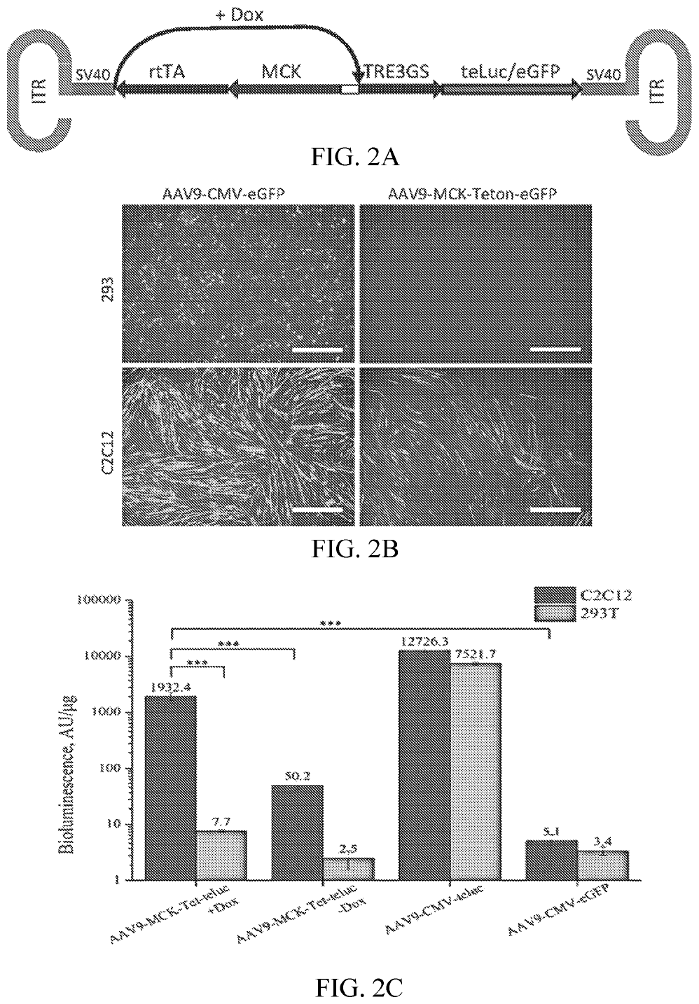

AAV9-P_{Mus}-TetOn-eGFP and AAV9-P_{Mus}-TetOn-teLuc viruses were prepared using 3 plasmids co-transfection method in 293T cells. AAV9-P_{CMV}-eGFP/teLuc viruses were used as constitutive expression control. Same amount of AAV9-MCK-TetOn-eGFP/teLuc and AAV9-CMV-eGFP/teLuc viruses were used to infect 293T and differentiated C2C12 cells. Dox (1 ug/ul) were added start from day 1 post-infection for the Pₘᵤₛ-TetOn groups. At day 3 post-infection, images were taken for GFP expression (FIG. 2B), and cells were lysed for luciferase activity assay if teLuc were used as reporter gene.

AAV9-CMV-eGFP expressed at high levels in both 293T and C2C12 cells, whereas AAV9-MCK-TetOn-eGFP had little or no expression in 293T cells but moderate GFP expression were observed in differentiated C2C12 cells in presence of Dox. To better quantify reporter gene expression levels in different cell lines, luciferase activity assays were conducted after AAV9-P_{CMV}-teLuc and AAV9-Pₘᵤₛ-TetOn-teLuc infection (FIG. 2C). In the Pₘᵤₛ-TetOn group (with Dox), luciferase activity was 250-fold higher in differentiated C2C12 cells than in 293T cells. In C2C12 cells, with the presence of Dox, the luciferase activity was 38-fold higher than without Dox induction. These results indicated that the AAV9-Pₘᵤₛ-TetOn vector can specifically express reporter gene in muscle cells and the expression can be regulated by Dox administration.

pAAV MCK TetOn3G eGFP (SEQ ID NO: 5):
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTAGATCTAACTTGTTTATTGCAG
CTTATAATGGTTACAAATAAGGCAATAGCATCACAAATTTCACAAATAAGGCATT
TTTTTCACTGCATTCTAGTTTTGGTTTGTCCAAACTCATCAATGTATCTTATCATGT
CTGGATCTTACCCGGGGAGCATGTCAAGGTCAAAATCGTCAAGAGCGTCAGCAG
GCAGCATATCAAGGTCAAAGTCGTCAAGGGCATCGGCTGGGAGCATGTCTAAGT
CAAAATCGTCAAGGGCGTCGGTCGGCCCGCCGCTTTCGCACTTTAGCTGTTTCTC
CAGGCCACATATGATTAGTTCCAGGCCGAAAAGGAAGGCAGGTTCGGCTCCCTG
CCGGTCGAACAGCTCAATTGCTTGTTTCAGAAGTGGGGGCATAGAATCGGTGGT
AGGTGTCTCTCTTTCCTCTTTTGCTACTTGATGCTCCTGTTCCTCCAATACGCAGC
CCAGTGTAAAGTGGCCCACGGCGGACAGAGCGTACAGTGCGTTCTCCAGGGAGA
AGCCTTGCTGACACAGGAACGCGAGCTGATTTTCCAGGGTTTCGTACTGTTTCTC
TGTTGGGCGGGTGCCGAGATGCACTTTAGCCCCGTCGCGATGTGAGAGGAGAGC
ACAGCGGTATGACTTGGCGTTGTTCCGCAGAAAGTCTTGCCATGACTCGCCTTCC
AGGGGGCAGGAGTGGGTATGATGCCTGTCCAGCATCTCGATTGGCAGGGCATCG
AGCAGGGCCCGCTTGTTCTTCACGTGCCAGTACAGGGTAGGCTGCTCAACTCCCA
GCTTTTGAGCGAGTTTCCTTGTCGTCAGGCCTTCGATACCGACTCCATTGAGTAAT
TCCAGAGCAGAGTTTATGACTTTGCTCTTGTCCAGTCTAGACATGGTAATTCGAT
GATCCCCCTTAATTAATGGGCCGCCGCCGGCCCCGGAGCCTTTTATCGAGGCGGG
CGGGAGCACCGCCCGGCCCCCAGGAATGCGGCCCCGGCCGAGGGCGGACGGGA
CACCCAAATATGGCGACGGCACCATTCCTCACCCGTCGCCATATTTCGGTGTCCA
CCATTCCTCACCGCTCTAAAAATAACTCCCCGCTCTAAAAATAACTCCCCCAACA
CCTGCTGCCTGCCCACCATTCCTCACCGCTCTAAAAATAACTCCCCACCATTCCTC
ACCCGTCGCCATATTTCGGTGTCGTGAGGAATGGTGCCGAGGGCGGACGGCCGC
ATGCATTTAAATACCAGGGACAGGGTTATTTTTAGAGCGAGCTTCTCCTCCATGG
TGTACAGAGCCTAAGACCCAGGCACCGGGGGGGGTAACCGCTCAGGCTCAGGC
AGCAGGTGTTGGGGGGGGGGGGGCAGCAGGTGTTGGGGTTAATTATAACCAGGC
ATCTCGGGTGTCCCCAGGCCTTGCCTCCTTACATGGGCAGCCTAGACCCGTAGTA
TTTAAATACCAGGGACAGGGTTATTTTTAGAGCGAGCTTCTCCTCCATGGTGTAC
AGAGCCTAAGACCCAGGCACCGGGGTGGGGTAACCGCTCAGGCTCAGGCAGCAG
GTGTTGGGGGGGGGGGGGCAGCAGGTGTTGGGGTTAATTATAACCAGGCATCTC
GGGTGTCCCCAGGCCTTGCCTCCTTACATGGGCAGCCTAGACCCGTAGTGGTTAA
TTAAAATAAAATATCTTTATTTTCATTACATCTGTGTGTTGGTTTTTTGTGTGAAT
CGATAGTACTAACATACGCTCTCCATCAAAACAAAACGAAACAAAACAAACTAG
CAAAATAGGCTGTCCCCAGTGCAAGTGCAGGTGCCAGAACATTTCTCTCTCGAGT
TTACTCCCTATCAGTGATAGAGAACGTATGAAGAGTTTACTCCCTATCAGTGATA
GAGAACGTATGCAGACTTTACTCCCTATCAGTGATAGAGAACGTATAAGGAGTTT
ACTCCCTATCAGTGATAGAGAACGTATGACCAGTTTACTCCCTATCAGTGATAGA
GAACGTATCTACAGTTTACTCCCTATCAGTGATAGAGAACGTATATCCAGTTTAC
TCCCTATCAGTGATAGAGAACGTATAAGCTTTGCTTATGTAAACCAGGGCGCCTA
TAAAAGAGTGCTGATTTTTTGAGTAAACTTCAATTCCACAACACTTTTGTCTTATA
CCAACTTTCCGTACCACTTCCTACCCTCGTAAAGAATTCTGCAGTCGACGGTACC
GCGGGCCCGGGATCCACCGGTCGCCACCATGGTGAGCAAGGGCGAGGAGCTGTT
CACCGGGGTGGTGCCCATCCTGGTCGAGCTGGACGGCGACGTAAACGGCCACAA
GTTCAGCGTGTCCGGCGAGGGCGAGGGCGATGCCACCTACGGCAAGCTGACCCT
GAAGTTCATCTGCACCACCGGCAAGCTGCCCGTGCCCTGGCCCACCCTCGTGACC
ACCCTGACCTACGGCGTGCAGTGCTTCAGCCGCTACCCCGACCACATGAAGCAG
CACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCG
ACACCCTGGTGAACCGCATCGAGCTGAAGGGCATCGACTTCAAGGAGGACGGCA
ACATCCTGGGGCACAAGCTGGAGTACAACTACAACAGCCACAACGTCTATATCA
TGGCCGACAAGCAGAAGAACGGCATCAAGGTGAACTTCAAGATCCGCCACAACA
TCGAGGACGGCAGCGTGCAGCTCGCCGACCACTACCAGCAGAACACCCCCATCG
GCGACGGCCCCGTGCTGCTGCCCGACAACCACTACCTGAGCACCCAGTCCGCCCT
GAGCAAAGACCCCAACGAGAAGCGCGATCACATGGTCCTGCTGGAGTTCGTGAC
CGCCGCCGGGATCACTCTCGGCATGGACGAGCTGTACAAGTAAAG
CGGCCGCACGCGTGGATCCACCGGTGATCCAGACATGATAAGATACATTGATGA
GTTTGGACAAACCAAAACTAGAATGCAGTGAAAAAAATGCCTTATTTGTGAAAT
TTGTGATGCTATTGCCTTATTTGTAACCATTATAAGCTGCAATAAACAAGTTGAT
ATCGATAGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCCCGGGC
GGCCTCAGTGAGCGAGCGAGCGCGCAGCTGCCTGCAGGCAATTCGTAATCATGG
TCATAGCTGTTTCCTGTGTGAAATTGTTATCCGCTCACAATTCCACACAACATAC
GAGCCGGAAGCATAAAGTGTAAAGCCTGGGGTGCCTAATGAGTGAGCTAACTCA
CATTAATTGCGTTGCGCTCACTGCCCGCTTTCCAGTCGGGAAACCTGTCGTGCCA
GCTGCATTAATGAATCGGCCAACGCGCGGGGAGAGGCGGTTTGCGTATTGGGCG
CTCTTCCGCTTCCTCGCTCACTGACTCGCTGCGCTCGGTCGTTCGGCTGCGGCGAG
CGGTATCAGCTCACTCAAAGGCGGTAATACGGTTATCCACAGAATCAGGGGATA
ACGCAGGAAAGAACATGTGAGCAAAAGGCCAGCAAAAGGCCAGGAACCGTAAA
AAGGCCGCGTTGCTGGCGTTTTTCCATAGGCTCCGCCCCCCTGACGAGCATCACA
AAAATCGACGCTCAAGTCAGAGGTGGCGAAACCCGACAGGACTATAAAGATACC
AGGCGTTTCCCCCTGGAAGCTCCCTCGTGCGCTCTCCTGTTCCGACCCTGCCGCTT
ACCGGATACCTGTCCGCCTTTCTCCCTTCGGGAAGCGTGGCGCTTTCTCATAGCTC
ACGCTGTAGGTATCTCAGTTCGGTGTAGGTCGTTCGCTCCAAGCTGGGCTGTGTG
CACGAACCCCCCGTTCAGCCCGACCGCTGCGCCTTATCCGGTAACTATCGTCTTG
AGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGC
CTAACTACGGCTACACTAGAAGAACAGTATTTGGTATCTGCGCTCTGCTGAAGCC
AGTTACCTTCGGAAAAAGAGTTGGTAGCTCTTGATCCGGCAAACAAACCACCGC
TGGTAGCGGTGGTTTTTTTGTTTGCAAGCAGCAGATTACGCGCAGAAAAAAAGG
ATCTCAAGAAGATCCTTTGATCTTTTCTACGGGGTCTGACGCTCAGTGGAACGAA
AACTCACGTTAAGGGATTTTGGTCATGAGATTATCAAAAAGGATCTTCACCTAGA
TCCTTTTAAATTAAAAATGAAGTTTTAAATCAATCTAAAGTATATATGAGTAAAC
TTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACG
GGAGGGCTTACCATCTGGCCCCAGTGCTGCAATGATACCGCGAGACCCACGCTC
ACCGGCTCCAGATTTATCAGCAATAAACCAGCCAGCCGGAAGGGCCGAGCGCAG
AAGTGGTCCTGCAACTTTATCCGCCTCCATCCAGTCTATTAATTGTTGCCGGGAA
GCTAGAGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCCGGTTCC
CAACGATCAAGGCGAGTTACATGATCCCCCATGTTGTGCAAAAAAGCGGTTAGC
TCCTTCGGTCCTCCGATCGTTGTCAGAAGTAAGTTGGCCGCAGTGTTATCACTCA
TGGTTATGGCAGCACTGCATAATTCTCTTACTGTCATGCCATCCGTAAGATGCTTT
TCTGTGACTGGTGAGTACTCAACCAAGTCATTCTGAGAATAGTGTATGCGGCGAC
CGAGTTGCTCTTGCCCGGCGTCAATACGGGATAATACCGCGCCACATAGCAGAA
CTTTAAAAGTGCTCATCATTGGAAAACGTTCTTCGGGGCGAAAACTCTCAAGGAT
CTTACCGCTGTTGAGATCCAGTTCGATGTAACCCACTCGTGCACCCAACTGATCT
TCAGCATCTTTTACTTTCACCAGCGTTTCTGGGTGAGCAAAAACAGGAAGGCAAA
ATGCCGCAAAAAAGGGAATAAGGGCGACACGGAAATGTTGAATACTCATACTCT
TCCTTTTTCAATATTATTGAAGCATTTATCAGGGTTATTGTCTCATGAGCGGATAC
ATATTTGAATGTATTTAGAAAAATAAACAAATAGGGGTTCCGCGCACATTTCCCC
GAAAAGTGCCACCTGACGTCTAAGAAACCATTATTATCATGACATTAACCTATAA
AAATAGGCGTATCACGAGGCCCTTTCGTCTCGCGCCAGCTGGCGAAAGGGGGAT
GTGCTGCAAGGCGATTAAGTTGGGTAACGCCAGGGTTTTCCCAGTCACGACGTTG
TAAAACGACGGCCAGTGCCAAGCTG
pAAV MCK TetOn3G IRESwt teLuc (SEQ ID NO: 6):
CCTGCAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAG
AGGGAGTGGCCAACTCCATCACTAGGGGTTCCTAGATCTAACTTGTTTATTGCAG
CTTATAATGGTTACAAATAAGGCAATAGCATCACAAATTTCACAAATAAGGCATT
TTTTTCACTGCATTCTAGTTTTGGTTTGTCCAAACTCATCAATGTATCTTATCATGT
CTGGATCTTACCCGGGGAGCATGTCAAGGTCAAAATCGTCAAGAGCGTCAGCAG
GCAGCATATCAAGGTCAAAGTCGTCAAGGGCATCGGCTGGGAGCATGTCTAAGT
CAAAATCGTCAAGGGCGTCGGTCGGCCCGCCGCTTTCGCACTTTAGCTGTTTCTC
CAGGCCACATATGATTAGTTCCAGGCCGAAAAGGAAGGCAGGTTCGGCTCCCTG
CCGGTCGAACAGCTCAATTGCTTGTTTCAGAAGTGGGGGCATAGAATCGGTGGT
AGGTGTCTCTCTTTCCTCTTTTGCTACTTGATGCTCCTGTTCCTCCAATACGCAGC
CCAGTGTAAAGTGGCCCACGGCGGACAGAGCGTACAGTGCGTTCTCCAGGGAGA
AGCCTTGCTGACACAGGAACGCGAGCTGATTTTCCAGGGTTTCGTACTGTTTCTC
TGTTGGGCGGGTGCCGAGATGCACTTTAGCCCCGTCGCGATGTGAGAGGAGAGC
ACAGCGGTATGACTTGGCGTTGTTCCGCAGAAAGTCTTGCCATGACTCGCCTTCC
AGGGGGCAGGAGTGGGTATGATGCCTGTCCAGCATCTCGATTGGCAGGGCATCG
AGCAGGGCCCGCTTGTTCTTCACGTGCCAGTACAGGGTAGGCTGCTCAACTCCCA
GCTTTTGAGCGAGTTTCCTTGTCGTCAGGCCTTCGATACCGACTCCATTGAGTAAT
TCCAGAGCAGAGTTTATGACTTTGCTCTTGTCCAGTCTAGACATGGTAATTCGAT
GATCCCCCTTAATTAATGGGCCGCCGCCGGCCCCGGAGCCTTTTATCGAGGCGGG
CGGGAGCACCGCCCGGCCCCCAGGAATGCGGCCCCGGCCGAGGGCGGACGGGA
CACCCAAATATGGCGACGGCACCATTCCTCACCCGTCGCCATATTTCGGTGTCCA
CCATTCCTCACCGCTCTAAAAATAACTCCCCGCTCTAAAAATAACTCCCCCAACA
CCTGCTGCCTGCCCACCATTCCTCACCGCTCTAAAAATAACTCCCCACCATTCCTC
ACCCGTCGCCATATTTCGGTGTCGTGAGGAATGGTGCCGAGGGCGGACGGCCGC
ATGCATTTAAATACCAGGGACAGGGTTATTTTTAGAGCGAGCTTCTCCTCCATGG
TGTACAGAGCCTAAGACCCAGGCACCGGGGTGGGGTAACCGCTCAGGCTCAGGC
AGCAGGTGTTGGGGGGGGGGGGGCAGCAGGTGTTGGGGTTAATTATAACCAGGC
ATCTCGGGTGTCCCCAGGCCTTGCCTCCTTACATGGGCAGCCTAGACCCGTAGTA
TTTAAATACCAGGGACAGGGTTATTTTTAGAGCGAGCTTCTCCTCCATGGTGTAC
AGAGCCTAAGACCCAGGCACCGGGGTGGGGTAACCGCTCAGGCTCAGGCAGCAG
GTGTTGGGGGGGGGGGGGCAGCAGGTGTTGGGGTTAATTATAACCAGGCATCTC
GGGTGTCCCCAGGCCTTGCCTCCTTACATGGGCAGCCTAGACCCGTAGTGGTTAA
TTAAAATAAAATATCTTTATTTTCATTACATCTGTGTGTTGGTTTTTTGTGTGAAT
CGATAGTACTAACATACGCTCTCCATCAAAACAAAACGAAACAAAACAAACTAG
CAAAATAGGCTGTCCCCAGTGCAAGTGCAGGTGCCAGAACATTTCTCTCTCGAGT
TTACTCCCTATCAGTGATAGAGAACGTATGAAGAGTTTACTCCCTATCAGTGATA
GAGAACGTATGCAGACTTTACTCCCTATCAGTGATAGAGAACGTATAAGGAGTTT
ACTCCCTATCAGTGATAGAGAACGTATGACCAGTTTACTCCCTATCAGTGATAGA
GAACGTATCTACAGTTTACTCCCTATCAGTGATAGAGAACGTATATCCAGTTTAC
TCCCTATCAGTGATAGAGAACGTATAAGCTTTGCTTATGTAAACCAGGGCGCCTA
TAAAAGAGTGCTGATTTTTTGAGTAAACTTCAATTCCACAACACTTTTGTCTTATA
CCAACTTTCCGTACCACTTCCTACCCTCGTAAAGAATTCGCGGCCGCACGCGTGG
ATCCGCCCCTCTCCCTCCCCCCCCCCTAACGTTACTGGCCGAAGCCGCTTGGAAT
AAGGCCGGTGTGCGTTTGTCTATATGTTATTTTCCACCATATTGCCGTCTTTTGGC
AATGTGAGGGCCCGGAAACCTGGCCCTGTCTTCTTGACGAGCATTCCTAGGGGTC
TTTCCCCTCTCGCCAAAGGAATGCAAGGTCTGTTGAATGTCGTGAAGGAAGCAGT
TCCTCTGGAAGCTTCTTGAAGACAAACAACGTCTGTAGCGACCCTTTGCAGGCAG
CGGAACCCCCCACCTGGCGACAGGTGCCTCTGCGGCCAAAAGCCACGTGTATAA
GATACACCTGCAAAGGCGGCACAACCCCAGTGCCACGTTGTGAGTTGGATAGTT
GTGGAAAGAGTCAAATGGCTCTCCTCAAGCGTATTCAACAAGGGGCTGAAGGAT
GCCCAGAAGGTACCCCATTGTATGGGATCTGATCTGGGGCCTCGGTGCACATGCT
TTACgTGTGTTTAGTCGAGGTTAAAAAACGTCTAGGCCCCCCGAACCACGGGGAC
GTGGTTTTCCTTTGAAAAACACGATGATAATATGGCCACAACCatggtcttcacactcgaaga
tttcgttggggactggcgacagacagccggctacaacttgagtcaagtccttgaacagggaggtgt
gtccagtttgtttcagaatctcggggtgtccgtaactccgatccaaaggattgtcctgagcggtga
aaatgggctgaagatcgacatccatgtcatcatcccgtatgaaggtctgagcggcgaccaaatggg
ccagatcgaaaaaatttttaaggtggtgtaccctgtggataatcatcactttaaggtgatcctgca
ctatggcacactggtaatcgacggggttacgccgaacatgatcgactatttcggacggccgtatga
aggcatcgccgtgttcgacggcaaaaagatcactgtaacagggaccctgtggaacggcaacaaaat
tatcgacgagcgcctgatcaaccccgacggctccctgctgttccgagtaaccatcaacggagtgac
cggctggcgtctgcatgaacgcattctggcgggctccggcgaacaaaaactcatctcagaagagga
tctgtaaACCGGTGATCCAGACATGATAAGATACATTGATGAGTTTGGACAAACCAAA
ACTAGAATGCAGTGAAAAAAATGCCTTATTTGTGAAATTTGTGATGCTATTGCCT
TATTTGTAACCATTATAAGCTGCAATAAACAAGTTGATATCGATAGGAACCCCTA
GTGATGGAGTTGGCCACTCCCTCTCTGCGCGCTCGCTCGCTCACTGAGGCCGGGC
GACCAAAGGTCGCCCGACGCCCGGGCTTTGCCCGGGCGGCCTCAGTGAGCGAGC
GAGCGCGCAGCTGCCTGCAGGCAATTCGTAATCATGGTCATAGCTGTTTCCTGTG
TGAAATTGTTATCCGCTCACAATTCCACACAACATACGAGCCGGAAGCATAAAG
TGTAAAGCCTGGGGTGCCTAATGAGTGAGCTAACTCACATTAATTGCGTTGCGCT
CACTGCCCGCTTTCCAGTCGGGAAACCTGTCGTGCCAGCTGCATTAATGAATCGG
CCAACGCGCGGGGAGAGGCGGTTTGCGTATTGGGCGCTCTTCCGCTTCCTCGCTC
ACTGACTCGCTGCGCTCGGTCGTTCGGCTGCGGCGAGCGGTATCAGCTCACTCAA
AGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCG
TTTTTCCATAGGCTCCGCCCCCCTGACGAGCATCACAAAAATCGACGCTCAAGTC
AGAGGTGGCGAAACCCGACAGGACTATAAAGATACCAGGCGTTTCCCCCTGGAA
GCTCCCTCGTGCGCTCTCCTGTTCCGACCCTGCCGCTTACCGGATACCTGTCCGCC
TTTCTCCCTTCGGGAAGCGTGGCGCTTTCTCATAGCTCACGCTGTAGGTATCTCAG
TTCGGTGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGTAAGAC
ACGACTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTACACTAG
AAGAACAGTATTTGGTATCTGCGCTCTGCTGAAGCCAGTTACCTTCGGAAAAAGA
GTTGGTAGCTCTTGATCCGGCAAACAAACCACCGCTGGTAGCGGTGGTTTTTTTG
TTTGCAAGCAGCAGATTACGCGCAGAAAAAAAGGATCTCAAGAAGATCCTTTGA
TCTTTTCTACGGGGTCTGACGCTCAGTGGAACGAAAACTCACGTTAAGGGATTTT
GGTCATGAGATTATCAAAAAGGATCTTCACCTAGATCCTTTTAAATTAAAAATGA
AGTTTTAAATCAATCTAAAGTATATATGAGTAAACTTGGTCTGACAGTTACCAAT
GCTTAATCAGTGAGGCACCTATCTCAGCGATCTGTCTATTTCGTTCATCCATAGTT
GCCTGACTCCCCGTCGTGTAGATAACTACGATACGGGAGGGCTTACCATCTGGCC
CCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATC
CGCCTCCATCCAGTCTATTAATTGTTGCCGGGAAGCTAGAGTAAGTAGTTCGCCA
GTTAATAGTTTGCGCAACGTTGTTGCCATTGCTACAGGCATCGTGGTGTCACGCT
CGTCGTTTGGTATGGCTTCATTCAGCTCCGGTTCCCAACGATCAAGGCGAGTTAC
ATGATCCCCCATGTTGTGCAAAAAAGCGGTTAGCTCCTTCGGTCCTCCGATCGTT
GTCAGAAGTAAGTTGGCCGCAGTGTTATCACTCATGGTTATGGCAGCACTGCATA
ATTCTCTTACTGTCATGCCATCCGTAAGATGCTTTTCTGTGACTGGTGAGTACTCA
ACCAAGTCATTCTGAGAATAGTGTATGCGGCGACCGAGTTGCTCTTGCCCGGCGT
CAATACGGGATAATACCGCGCCACATAGCAGAACTTTAAAAGTGCTCATCATTG
GAAAACGTTCTTCGGGGCGAAAACTCTCAAGGATCTTACCGCTGTTGAGATCCAG
TTCGATGTAACCCACTCGTGCACCCAACTGATCTTCAGCATCTTTTACTTTCACCA
GCGTTTCTGGGTGAGCAAAAACAGGAAGGCAAAATGCCGCAAAAAAGGGAATA
AGGGCGACACGGAAATGTTGAATACTCATACTCTTCCTTTTTCAATATTATTGAA
GCATTTATCAGGGTTATTGTCTCATGAGCGGATACATATTTGAATGTATTTAGAA
AAATAAACAAATAGGGGTTCCGCGCACATTTCCCCGAAAAGTGCCACCTGACGT
CTAAGAAACCATTATTATCATGACATTAACCTATAAAAATAGGCGTATCACGAG
GCCCTTTCGTCTCGCGCCAGCTGGCGAAAGGGGGATGTGCTGCAAGGCGATTAA
GTTGGGTAACGCCAGGGTTTTCCCAGTCACGACGTTGTAAAACGACGGCCAGTG
CCAAGCTG

### Example 4: Testing Recombinant AAV Vectors In Vivo

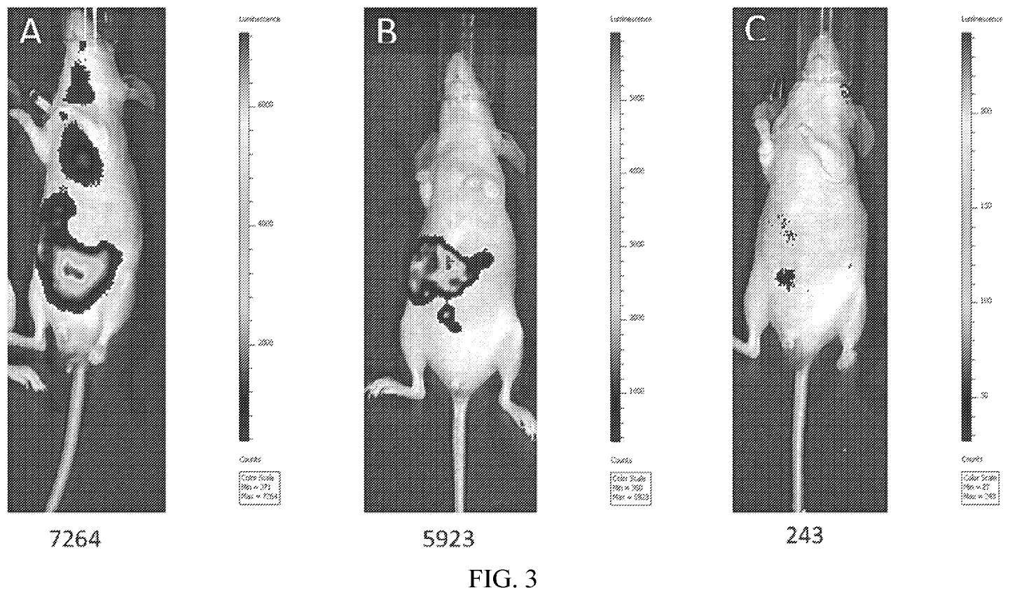

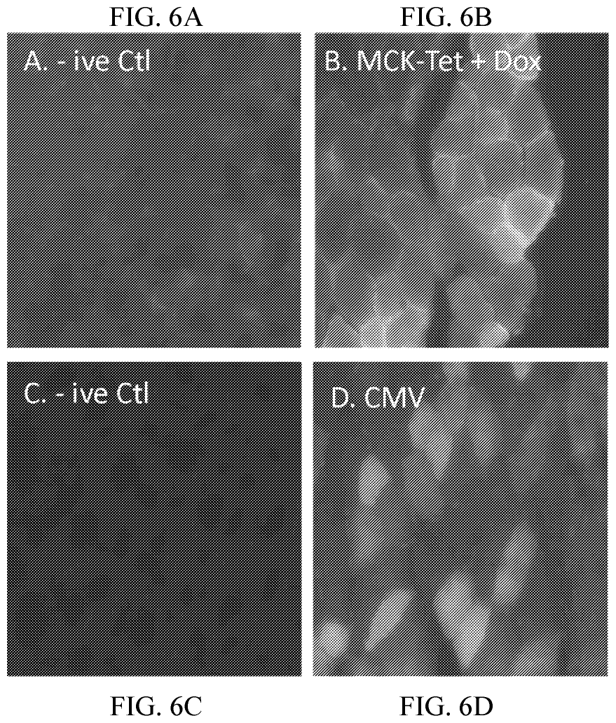

Similar to the in vitro tests, animals were divided into three groups: P_{CMV} control, Pₘᵤₛ-TetOn+Dox, and Pₘᵤₛ-TetOn-Dox, which were infected by AAV9-P_{CMV}-teLuc and AAV9-Pₘᵤₛ-TetOn-teLuc with and without Dox respectively. C57/B6 hairless mice were injected intravenously with 2×10¹² viral vectors, in Pₘᵤₛ-TetOn+Dox group, Dox diet were given two days post viral injection. One month after virus injection and/or Dox administration, whole body images were taken by the in vivo imaging system (IVIS) for overall luciferase expression level. CMV control group displayed strong bioluminescence from the abdomen area (FIG. 3A), Pₘᵤₛ-TetOn+Dox group had 80% bioluminescence level of the CMV group (FIG. 3B). Without Dox administration, the bioluminescence was only 4% compared to the induction group (FIG. 3C), which was considered as basal level expression. AAV9-P_{CMV}-eGFP and AAV9-Pₘᵤₛ-TetOn-eGFP were also injected to the animal intramuscularly to visualize the reporter gene expression in muscle tissue sections (FIG. 6). These results indicated that the Pₘᵤₛ-TetOn system can express gene of interest in vivo and gene expression can be switched on and off by administration and withdrawal of doxycycline.

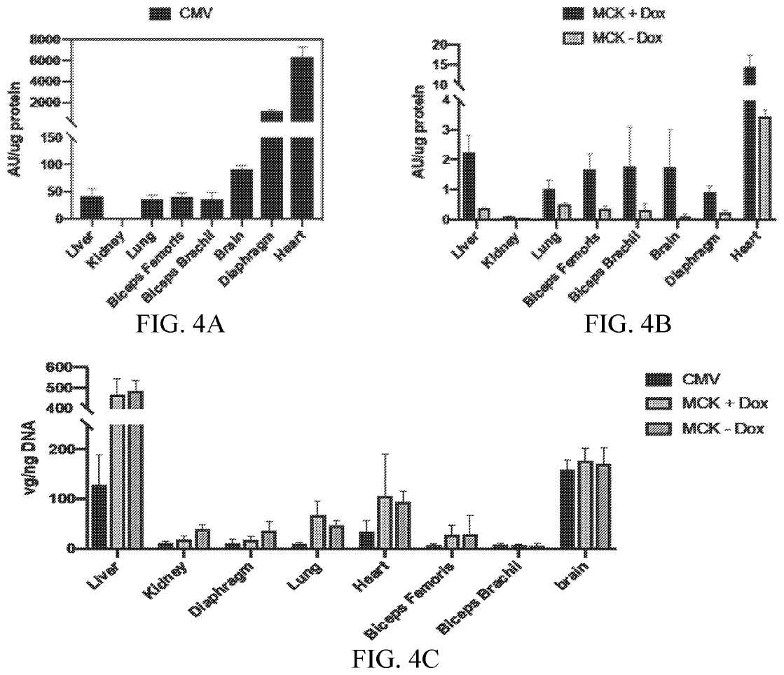

To test the tissue specificity of reporter gene expression, mice were sacrificed one month post virus injection. Heart, diaphragm, liver, kidney, lung, brain, biceps brachii and biceps femoris tissues were collected after IVIS imaging. As showed in FIG. 4A, the CMV control group showed highest expression level in the heart tissue, diaphragm and brain tissues also had strong luciferase activity. Skeletal muscles (biceps brachii and biceps femoris), lung, and liver had mild expression level, but in kidney, little luciferase activity was detected. In the MCK-TetOn+Dox group, the overall tissue expression levels were less efficient than the CMV group (FIG. 4B). The highest expression level was found in heart, skeletal muscles, brain, and liver had less reporter gene expression. And similar to the CMV group, kidney tissues luciferase expression was hard to detect.

To further investigate the tissue specific expression of our AAV9 vector, we tested the actual virus number in selected tissues. From the dissected tissues mentioned above, tissue genomic and viral DNAs were extracted, viral copy number were determined by qPCR and then normalized by genomic DNA concentration, the results are shown in FIG. 4C. In all three groups, the overall tissue distributions are similar. Liver and brain are the two main target for AAV9. The MCK groups showed higher distribution of the viruses to heart, lung, and biceps femoris compared to the CMV group, however, the differences between 3 groups were not statistically significant.

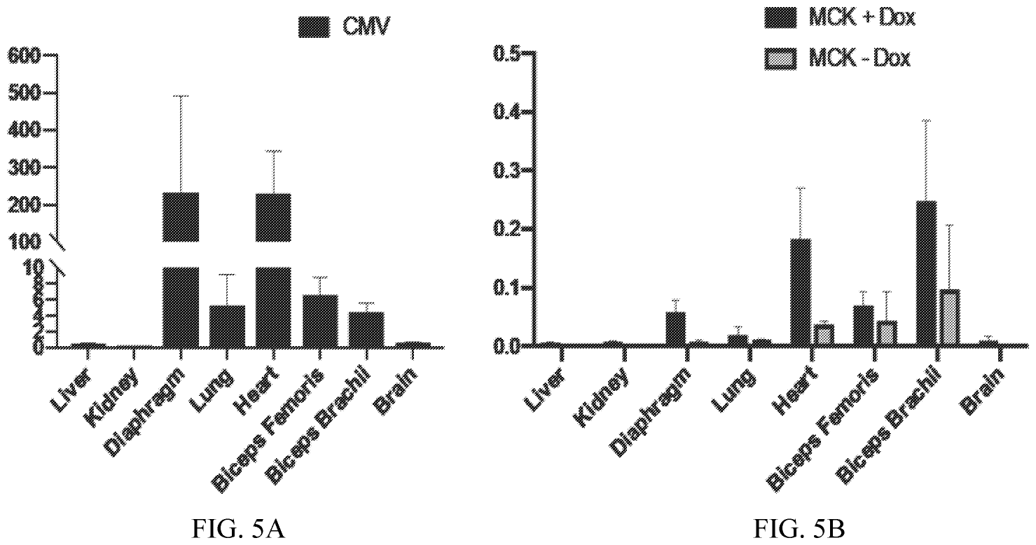

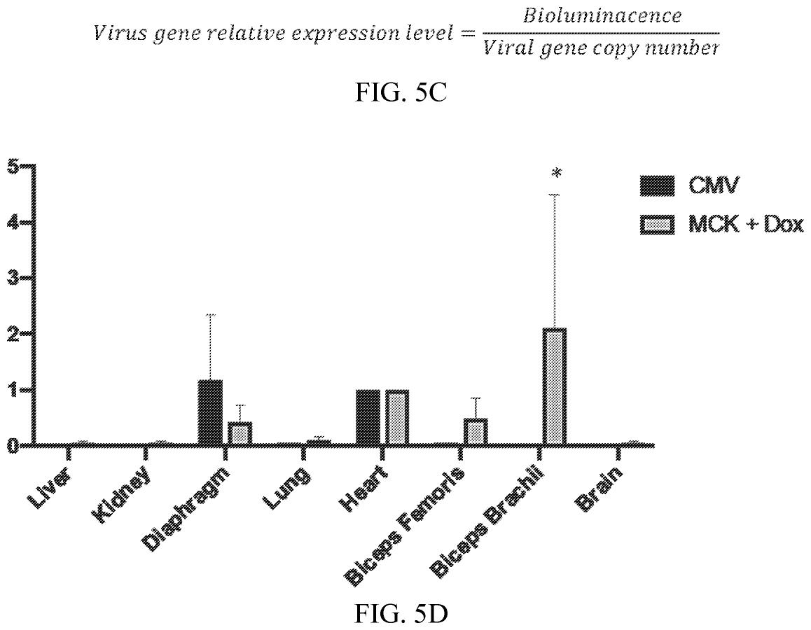

Virus distributions were observed to be different among different tissues, and thus the luciferase expression level difference among the tissues may be caused by various virus amount. The actual reporter gene expression was further analyzed based on the gene copy number in the specific tissues. The virus gene relative expression level was defined as the bioluminescence from the tissue luciferase assay divided by viral gene copy number (normalized by genomic DNA concentration) in the same piece of tissue (FIG. 5C). In the CMV group, virus gene relative expression level was found the to be highest in diaphragm and heart tissue (FIG. 5A), however in the MCK-TetOn+Dox group, the highest expression levels were observed in biceps femoris and heart (FIG. 5B).

The relative expression in heart was set to 1 to allow comparison of expression in different tissues between the P_{CMV} and Pₘᵤₛ-TetOn groups. Despite the overall higher expression level driven by the P_{CMV} promoter, the Pₘᵤₛ promoter can drive stronger expression in the muscle tissues (FIG. 5D). In the Pₘᵤₛ-TetOn+Dox group, relative expressions were stronger in the biceps branchii and biceps femoris muscle than in the P_{CMV} group. On the other hand, the P_{CMV} group had higher level of relative expression in the diaphragm tissue than the Pₘᵤₛ-TetOn group. These results showed that the AAV9-Pₘᵤₛ-TetOn-teLuc vector can express reporter gene at a high level in skeletal muscles.

### Example 5: Assessing Long-Term Gene Expression and Repeat Induction by the Recombinant AAV Vector

The studies presented in the present disclosure have demonstrated that the AAV9-MCK-TetOn-eGFP/teLuc vector can be turned on and off, however, whether the expression can be modulated multiple times remain unknown. To address this question, AAV9-MCK-TetOn-teLuc (2×10¹² vg/animal) viruses were injected to the hairless mice through intravenous (I.V.) route. After virus injection and dox induction, in vivo luciferase activity was monitored via in vivo imaging system (IVIS), beginning 2 weeks post-induction (basal expression was imaged one day post virus injection); after that, dox was taken off and continuous luciferase expressions were tracked biweekly until dropping back to basal level. Expressions were induced for three times. In the I.V. injection group, Dox diet initiated gene expression, and the expression level dropped to basal level after 6 weeks. Second induction drove the gene expression to a comparable level after the first induction, and the third induction didn't obviously decrease the expression level. Each Dox withdrawal took 6 weeks to decrease expression to the basal level.

### Example 6: Selected Discussion

In the present disclosure, an AAV vector is described which possesses both spatial and temporal control of gene expression. The Pₘᵤₛ hybrid enhancer/promoter can drive gene expression specifically in skeletal muscles, and the TetOn system can serve as an expression switch.

The promoter and enhancer of human cytomegalovirus (CMV) has been identified as one of the most potent DNA elements to drive transgene expression in variety of mammalian cells. However, extensive gene expression can cause to strong immune responses and unwanted side effects. The studies of the present disclosure aimed to develop a muscle-specific expression AAV vector for muscle and neuromuscular diseases. The construct disclosed herein made use of 2 copies of the Mck enhancer elements ligated at the upstream of the synthetic muscle promoter, in conjunction with the TetOn system to achieve both spatial and temporal control of gene expression.

The tetracycline agent-controlled transcription system is widely used for inducible gene expression. It was first developed as an expression silencing tool (TetOff) and was later modified to achieve the reverse function (TetOn) by random mutagenesis screening. However, the mutation also caused significantly reduced sensitivity towards the effector doxycycline. The TetOn system was then further improved to lower background expression and enhance the sensitivity to doxycycline.

The MCK enhancer/synthetic muscle promoter was cloned together with the TetOn3G system into AAV9 vector. This recombinant vector can express gene of interest in high levels in C2C12 myotubes than in HEK 293T cells, and the expression can be activated by Dox. In vivo experiments showed higher relative expression level in skeletal muscles from the MCK-TetOn vector compared to the CMV vector. Repeatable induction has also been verified after 3 rounds of Dox administration/withdrawal, and the expression would drop to the basal level after 6 weeks. This indicated a maximum time frame of the induction interval.

### Enumerated Embodiments

The following enumerated embodiments are provided, the numbering of which is not to be construed as designating levels of importance.

Embodiment 1 provides a recombinant AAV vector comprising a payload transgene, wherein the payload transgene is operably linked to an inducible transcriptional activator, which is operably linked to a muscle-specific promoter, wherein the recombinant AAV vector further comprises a capsid gene specific for muscle tissue.

Embodiment 2 provides the recombinant AAV vector of Embodiment 1, wherein the muscle-specific promoter is a hybrid muscle-specific promoter.

Embodiment 3 provides the recombinant AAV vector of Embodiment 2, wherein the hybrid muscle-specific promoter is a modified syn promoter.

Embodiment 4 provides the recombinant AAV vector of Embodiment 3, wherein the modified syn promoter comprises a Mck enhancer.

Embodiment 5 provides the recombinant AAV vector of Embodiment 1, wherein the inducible transcriptional activator is a tetracycline agent-inducible promoter.

Embodiment 6 provides the recombinant AAV of Embodiment 5, wherein the tetracycline agent is selected from the group consisting of tetracycline, doxycycline, and anhydrotetracycline.

Embodiment 7 provides the recombinant AAV of Embodiment 1, wherein the capsid gene specific for muscle tissue is based on an AAV9 capsid gene.

Embodiment 8 provides an isolated polynucleotide comprising an AAV vector, the vector comprising a payload transgene, wherein the payload transgene is operably linked to an inducible transcriptional activator, which is operably linked to a muscle-specific promoter, wherein the AAV vector further comprises a capsid gene specific for muscle tissue.

Embodiment 9 provides the isolated polynucleotide of Embodiment 8, wherein the muscle-specific promoter is a hybrid muscle-specific promoter.

Embodiment 10 provides the isolated polynucleotide of Embodiment 9, wherein the hybrid muscle-specific promoter is a modified syn promoter.

Embodiment 11 provides the isolated polynucleotide of Embodiment 8, wherein the modified syn promoter comprises a Mck enhancer.

Embodiment 12 provides the isolated polynucleotide of Embodiment 8, wherein the inducible transcriptional activator is a tetracycline agent-inducible promoter.

Embodiment 13 provides the isolated polynucleotide of Embodiment 12, wherein the tetracycline agent is selected from the group consisting of tetracycline, doxycycline, and anhydrotetracycline.

Embodiment 14 provides the isolated polynucleotide of Embodiment 8, wherein the capsid gene specific for muscle tissue is based on an AAV9 capsid gene.

Embodiment 15 provides a composition comprising the recombinant AAV vector of any one of Embodiments 1-7.

Embodiment 16 provides a method of introducing a transgene into a target cell, comprising:
i. contacting an immortalized cell with the recombinant AAV vector of any one of Embodiments 1-7 and one or more helper plasmids, thereby producing a packaging cell;
ii. culturing the packaging cell to produce AAV vector particles;
iii. isolating and purifying at least a fraction of the AAV vector particles; and
iv. contacting the target cell with an effective amount of the AAV vector particles, thereby introducing the transgene into the target cell; and
v. treating the target cell with an effective amount of a tetracycline agent thereof.

Embodiment 17 provides the method of Embodiment 16, wherein the target cell is a muscle cell.

Embodiment 18 provides the method of Embodiment 16, wherein the tetracycline agent is selected from the group consisting of tetracycline, doxycycline, and anhydrotetracycline.

Embodiment 19 provides the method of Embodiment 16, wherein the immortalized cell is selected from the group consisting of a HEK293 cell, a HEK293 cell lacking a large T element, a HeLa cell, a CHO cell, and a hTERT-immortalized cell.

Embodiment 20 provides a method of treating, ameliorating, and/or preventing a disease in a subject in need thereof, the method comprising administering to the subject an effective amount of the composition of Embodiment 15 and further administering to the subject an effective amount of a tetracycline agent.

Embodiment 21 provides the method of Embodiment 20, wherein expression of the transgene corrects and/or ameliorates dysfunction of an endogenous gene.

Embodiment 22 provides the method of Embodiment 20, wherein the disease is related to dysfunction of an endogenous gene.

Embodiment 23 provides the method of Embodiment 20, wherein the tetracycline agent is selected from the group consisting of tetracycline, doxycycline, and anhydrotetracycline.

Embodiment 24 provides the method of Embodiment 20, wherein the subject is a mammal.

Embodiment 25 provides the method of Embodiment 20, wherein the subject is a human.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A recombinant AAV vector comprising a payload transgene,
wherein the payload transgene is operably linked to an inducible transcriptional activator, which is operably linked to a muscle-specific promoter,
wherein the recombinant AAV vector further comprises a capsid gene specific for muscle tissue.

2. The recombinant AAV vector of claim 1, wherein the muscle-specific promoter is a hybrid muscle-specific promoter.

3. The recombinant AAV vector of claim 2, wherein the hybrid muscle-specific promoter is a modified syn promoter.

4. The recombinant AAV vector of claim 3, wherein the modified syn promoter comprises a Mck enhancer.

5. The recombinant AAV vector of claim 1, wherein the inducible transcriptional activator is a tetracycline agent-inducible promoter.

6. The recombinant AAV of claim 5, wherein the tetracycline agent is selected from the group consisting of tetracycline, doxycycline, and anhydrotetracycline.

7. The recombinant AAV of claim 1, wherein the capsid gene specific for muscle tissue is based on an AAV9 capsid gene.

8. An isolated polynucleotide encoding an AAV vector, the vector comprising a payload transgene,
wherein the payload transgene is operably linked to an inducible transcriptional activator, which is operably linked to a muscle-specific promoter,
wherein the AAV vector further comprises a capsid gene specific for muscle tissue.

9. The isolated polynucleotide of claim 8, wherein the muscle-specific promoter is a hybrid muscle-specific promoter.

10. The isolated polynucleotide of claim 9, wherein the hybrid muscle-specific promoter is a modified syn promoter.

11. The isolated polynucleotide of claim 10, wherein the modified syn promoter comprises a Mck enhancer.

12. The isolated polynucleotide of claim 8, wherein the inducible transcriptional activator is a tetracycline agent-inducible promoter.

13. The isolated polynucleotide of claim 12, wherein the tetracycline agent is selected from the group consisting of tetracycline, doxycycline, and anhydrotetracycline.

14. The isolated polynucleotide of claim 8, wherein the capsid gene specific for muscle tissue is based on an AAV9 capsid gene.

15. A composition comprising the recombinant AAV vector of claim 1.

16. A method of introducing a transgene into a target cell, comprising:
i. contacting an immortalized cell with the recombinant AAV vector of claim 1 and one or more helper plasmids, thereby producing a packaging cell;
ii. culturing the packaging cell to produce AAV vector particles;
iii. isolating and purifying at least a fraction of the AAV vector particles; and
iv. contacting the target cell with an effective amount of the AAV vector particles, thereby introducing the transgene into the target cell; and
V. treating the target cell with an effective amount of a tetracycline agent thereof.

17. The method of claim 16, wherein at least one of the following applies:
(a) the immortalized cell is selected from the group consisting of a HEK293 cell, a HEK293 cell lacking a large T element, a HeLa cell, a CHO cell, and a hTERT-immortalized cell;
(b) the target cell is a muscle cell.

18. The method of claim 16, wherein the tetracycline agent is selected from the group consisting of tetracycline, doxycycline, and anhydrotetracycline.

19. (canceled)

20. A method of treating, ameliorating, or preventing a disease in a subject in need thereof, the method comprising administering to the subject an effective amount of a composition comprising the recombinant AAV vector of claim 1 and further administering to the subject an effective amount of a tetracycline agent.

21. The method of claim 20, wherein at least one of the following applies:
(a) expression of the transgene corrects and/or ameliorates dysfunction of an endogenous gene;
(b) the disease is related to dysfunction of an endogenous gene;
(c) the tetracycline agent is selected from the group consisting of tetracycline, doxycycline, and anhydrotetracycline;
(d) the subject is a mammal;
(e) the subject is human.

22-25. (canceled)
